(19) United States
(12) Patent Application Publication
(10) Pub. No.: US 2011/0282426 A1
(43) Pub. Date: Nov. 17, 2011

(54) ENDOVASCULAR DEVICES AND ASSOCIATED SYSTEMS AND METHODS

(76) Inventors: Ashish Sudhir Mitra, New South Wales (AU); Martin Kean Chong Ng, New South Wales (AU); Michacl Skalsky, New South Wales (AU)

(21) Appl. No.: 13/145,957

(22) PCT Filed: Jan. 20, 2010
(86) PCT No.: PCT/AU2010/000050

§ 371 Date: Jul. 22, 2011

Related U.S. Application Data
(60) Provisional application No. 61/147,036, filed on Jan. 23, 2009.

(51) Int. Cl. A61F 2/84; A61M 25/10

(52) U.S. Cl. 623/1.11; 604/103.02

(57) ABSTRACT

An endoluminal device having a reduced delivery profile for delivery through a lumen and a greater released profile for placement in the lumen. The reduced profile configuration allows the compact delivery of agents or other components of a delivery system to a target site.

### FIELD OF THE INVENTION

The present disclosure is directed generally to endoluminal devices and associated systems and methods. Several aspects of the present disclosure, more specifically, are directed to anchoring of an endoluminal prosthesis to a vessel wall.

### BACKGROUND

An aneurysm is a localized, blood-filled dilation of a blood vessel caused by disease or weakening of the vessel wall. Aneurysms affect the ability of the vessel to conduct fluids, and can be life threatening if left untreated. Aneurysms most commonly occur in arteries at the base of the brain and in the aorta. As the size of an aneurysm increases, there is an increased risk of rupture, which can result in severe hemorrhage or other complications including sudden death.

Aneurysms are typically treated by surgically removing a part or all of the aneurysm and implanting a replacement prosthetic section into the body lumen. Such procedures, however, can require extensive surgery and recovery time. Patients often remain hospitalized for several days following the procedure, and can require several months of recovery time. Moreover, the morbidity and mortality rates associated with such major surgery can be significantly high.

Another approach for treating aneurysms involves deployment of an endovascular graft assembly at the affected site. Such procedures typically include intravascular delivery of the endovascular graft assembly to the site of the aneurysm. The graft is then expanded or deployed in situ and the ends of the graft are anchored to the body lumen on each side of the aneurysm. In this way, the graft effectively excludes the aneurysm sac from circulation.

One concern with many conventional endovascular graft assemblies, however, is the long term durability of such structures. Over time, for example, the graft can become separated from an inner surface of the body lumen, and such separation can result in endoleaks. As used herein, endoleak is defined as a persistent blood or other fluid flow outside the lumen of the endoluminal graft, but within the aneurysm sac or adjacent vascular segment being treated by the device. When an endoleak occurs, it can cause continuous pressurization of the aneurysm sac and may result in an increased risk of rupture.

In addition to endoleaks, another concern with many conventional endovascular graft assemblies is the delivery of endoluminal reactants to such devices. For example, after a practitioner has found an optimal location for the graft, the device must be fixed to the wall of the body lumen and fully sealed at each end of the graft to prevent endoleaks and achieve a degree of fixation that will prevent subsequent device migration and/or dislodgement.

### SUMMARY OF THE INVENTION

In a first aspect, the present invention provides an endoluminal device for delivering an agent to a vessel of a subject, said endoluminal device comprising:

at least one flexible support member configured for placement at least partially between an endoluminal prosthesis and a wall of a body lumen;

at least one agent carried by the support member;

said support member being changeable between a first relatively reduced radial configuration and a second relatively increased radial configuration;

wherein in said first reduced radial configuration, the support member comprises an elongate member having a length which extends a distance from a first end to a second end; and

wherein in said second increased radial configuration, said distance between said first end and said second end is relatively reduced.

### DESCRIPTION OF EMBODIMENTS OF THE INVENTION

In another aspect, the present invention provides an endoluminal assembly including:

at least one support member;

at least one agent carried by said support member, wherein said support member is changeable between a first relatively reduced radial configuration and a second relatively increased radial configuration; and

wherein in said first reduced radial configuration, the support member comprises an elongate member having a length which extends a distance from a first end to a second end; and wherein in said second increased radial configuration, said distance between said first end and said second end is relatively reduced;

said assembly further including a delivery means configured to hold said support member in said first reduced radial configuration, said delivery means also configured to deliver said endoluminal prosthesis to a target site in a vessel;

wherein said at least one support member of the assembly is configured for placement at least partially between said endoluminal prosthesis and a wall of a body lumen.

In a still further aspect, there is provided a method for delivering an agent between an endoluminal prosthesis and a wall of a body lumen, the method comprising:

advancing a sealing device to a desired location in the body lumen, said sealing device comprising a support member and at least one agent carried by the support member;

causing or allowing said support member to change from a first relatively reduced radial configuration to a second relatively increased radial configuration, wherein in said second increased radial configuration said support member defines a receiving region to receive at least a portion of the endoluminal prosthesis;

advancing the endoluminal prosthesis to a desired location wherein at least part of the prosthesis is received in said receiving region of said support member;

positioning an expandable member within a lumen of the endoluminal prosthesis and radially expanding the expandable member to exert a force on said support member; wherein said force causes the release of said agent from said support member.

In a still further aspect, there is provided a method for delivering an agent between an endoluminal prosthesis and a wall of a body lumen, the method comprising:

advancing the endoluminal prosthesis to a desired location in the body lumen, wherein the endoluminal prosthesis includes a sealing device positioned between the prosthesis and the wall of the body lumen, and wherein the sealing device includes (a) a support member including a shape memory material, and (b) a capsule carried by the support member;

positioning an expandable balloon in the body lumen with the sealing device between the balloon and the wall of the body lumen; and radially expanding the balloon to press the sealing device against the wall of the body lumen until the capsule releases an agent contained within the capsule.

### Release of Agent

The agent may be released when the support member is in its second increased radial configuration. Further, the release of the agent may be caused by the change of configuration of the support member.

Alternatively, the agent may be released after the change of configuration of said support member. The agent may not be released until the support member is subjected to a pressure. The pressure may be caused by the inflation of a balloon within said endoluminal prosthesis to cause an outward radial pressure.

Particularly, the agent may be held in a capsule of the support member whereupon the pressure exerted from a balloon expanding is sufficient to rupture the walls of the capsule to release the agent therefrom.

In a further embodiment, at least part of the capsule wall may be made from a degradable material. Once in situ, the wall degrades such that the agent held therein is released. This embodiment may be particularly useful when delivering agents that are to be slowly released over a period of time. Examples of degradable material include enzymatically degradable material, photo or UV degradable material or thermally degradable material.

In addition to the release of agent via the application of pressure, there are many other mechanisms to achieve said release. For example, the agent may be impregnated in the support member such that it is released over a period of time into the surrounding environment.

The agent may be held in a coating on the support member such that it is releasable therefrom.

Furthermore, the agent may be held in a capsule which, rather than rupture upon the application of pressure, has a frangible region in a wall of the capsule which may be broken by a user. Upon breaking of the wall, the agent may be released. It is envisaged that the frangible region may be broken by the use of a rip cord configuration or the like. The cord may extend from the region of placement of the device to a user. Pulling the rip cord breaks the capsule wall and releases the agent.

The agent may comprise a photo-curable substance in a relatively solid state upon introduction of the device into the body. Once in situ, the agent is subjected to photo-activation to cause it to change to a different and relatively less solid state. In the embodiment wherein the agent is an adhesive, the change in state to a relatively less solid state allows the adhesive to bind to the walls of the vessel and hold the endoluminal device thereto.

Similarly, the agent may comprise a thermo-curable agent. In this embodiment, the agent may change from a relatively solid state for introduction into the body to a relatively less solid state when in situ as a result of a relative change in temperature from outside the body to the temperature in situ.

In said embodiment wherein the support member is impregnated with said agent for release therefrom, the agent may be held in substantially closed pores within the material of the support member. Upon movement of the support member from the first to the second configuration, the pores may open to an outer surface of the support member to release said agent.

Further, the release mechanism may include an osmotic pressure differential.

In another embodiment, the one or more agent may be sheathed for delivery to a target site. Once positioned at the target site, the one or more agent may be unsheathed to enable release to the surrounding environment. This embodiment may have particular application for solid or semi-solid state agents.

In embodiments when the support member includes a capsule, the capsule may comprise a single annular compartment within the support member. In this embodiment, when the support member is in its second increased radial configuration, the capsule extends completely around the periphery of the endoluminal prosthesis. Alternatively, the capsule may only partially extend around the periphery of the prosthesis. Two or more capsules may extend around the prosthesis.

In other embodiments, the capsule may be segmented to include one or more compartments. The compartments may be relatively closely spaced. Further, the distance between adjacent compartments may vary.

The segmented capsule of this embodiment may not extend completely around the endoluminal prosthesis when the support member is in its second increased radial configuration.

In one embodiment wherein the support member includes a capsule said capsule may be substantially surrounded by said support member. In other embodiments, however, the capsule may be only partially enveloped by said support member.

Said capsule may comprise an outer wall to hold the agent therein. The outer wall may be made of a suitably flexible and biocompatible material. Alternatively, the capsule may comprise a more rigid structure having a pre-designed failure mechanism to allow the release of agent therefrom. Examples of suitable materials include but are not limited to low density polyethylene, high density polyethylene, polypropylene, polytetrafluoroethylene, silicone, or fluorosilicone. Other fluoropolymers that may be used for the construction of the capsule include: polytetrafluoroethylene, perfluoroalkoxy polymer resin, fluorinated ethylene-propylene, polyethylenetetrafluoroethylene, polyvinylfluoride, ethylenechlorotrifluoroethylene, polyvinylidene fluoride, polylychlorotrifluoroethylene, perfluoropolyether, fluorinated ethylene propylene, terpolymer of tetrafluoroethylene, hexafluoropropylene and vinylidene fluoride), polysulphone and polyether ether ketone (PEEK). It may also comprise non-polymeric materials such as glass, bioglass, ceramic, platinum and titanium. It may further comprise biologically based materials such as crosslinked collagen or alginates. It will be appreciated that the foregoing list is provided merely as an example of suitable materials and is not an exhaustive list. The capsule may be composed of a material or combination of materials different from those provided above.

The support member itself may be impregnated with the agent. The support member may further comprise individual depots of agent connected to or impregnated in an outer surface thereof.

In one embodiment wherein the support member includes one or more capsules, the agent may be released by rupturing of the capsule. As noted above, such rupture may be achieved by subjecting the capsule to a pressure. Typically, the capsule is subjected to a radial pressure.

Whether the agent is held in capsules, depots, in a coating or impregnated in the material of the support member, a number of different agents may be released from said support member.

For example, in an embodiment wherein the support member includes a capsule, the capsule may comprise an annular compartment divided by a frangible wall to separate the compartment into two or more sub-compartments. A different agent may be held in each sub-compartment. In one embodiment, the annular compartment may be divided longitudinally with at least one inner sub-compartment and at least one outer sub-compartment. Alternatively, the capsule may be divide radially into two or more sub-compartments. The sub-compartments may be concentric relative to one another.

In the embodiment wherein the capsule is segmented, the different compartments may hold different agents therein.

The rate of release of the agent from the support member may vary. As noted, in some embodiments, pressure exerted on said support member to rupture a capsule may release one or more agents. This rate of almost immediate release is particularly useful for delivering adhesive agents to a vessel to affix a prosthesis to a wall of said vessel.

However, it is envisaged that other agents may be released at a slower or at least a variable rate. Further, said agents may be released after the initial release of a primary agent (e.g. the adhesive).

For example, in an embodiment wherein the support member includes a segmented capsule, the first agent to be released may be held in one or more “immediate release” sub-compartments which comprise an outer wall configured to rupture under a pre-defined initial pressure. The support member may comprise one or more slow release sub-compartments having outer walls configured to withstand said initial pressure but which either rupture when subjected to a greater pressure or, alternatively which do not rupture but rather degrade over a certain period of time to release an agent held therein.

Typically, the capsule is configured to rupture to release one or more agents at a predetermined range of pressures. The range of rupture pressures includes between 5 and 250 psi. In an embodiment, the pressure range is between 5 and 125 psi. In a further embodiment, the pressure range is between 10 and 75 psi. In a still further embodiment, the pressure at which rupture occurs is approximately 50 psi.

The agent may further comprise a component of a graft assembly of other endoluminal assembly wherein said component is carried to a target site by the support member.

The support member may include a conformable band of material. In this embodiment, the material of the conformable band may be sufficiently flexible to conform to irregularities between the endoluminal prosthesis and a vessel wall. The band of material may comprise a mesh-like structure to catch released agents therein. This embodiment has the advantage of reducing embolisation of the agent from the target site in a vessel.

In said second reduced radial configuration, the support member may comprise a generally ring-like structure. In said second configuration the ring-like structure is configured to receive at least a portion of an endoluminal prosthesis such that it is positioned between said portion of the prosthesis and a vessel wall.

In a further embodiment, when said support member is in the second reduced radial configuration it may form a substantially helical configuration. The helical structure of the support member provides an internal passage therein to receive at least a portion of an endoluminal prosthesis.

The support member may include a shape memory material. The shape memory material may comprise one or more shape memory alloys. In this embodiment, movement of the shape memory material in a pre-determined manner causes the support member to move from said first reduced radial configuration to said second increased radial configuration.

The shape memory material may comprise Nickel-Titanium alloy (Nitinol). Alternatively, the shape memory material may comprise alloys of any one of the following combinations of metals: Copper-Zinc-Aluminium, Copper-Aluminium-Nickel, Copper-Aluminium-Nickel, Iron-Manganese-Silicon-Chromium-Manganese and Copper-Zirconium. Additionally, Titanium-Palladium-Nickel, Nickel-Titanium-Copper, Gold-Cadmium, Iron-Zinc-Copper-Aluminium, Titanium-Niobium-Aluminium, Uranium-Niobium, Hafnium-Titanium-Nickel, Iron-Manganese-Silicon, Nickel-Iron-Zinc-Aluminium, Copper-Aluminium-Iron, Titanium-Niobium, Zirconium-Copper-Zinc, Nickel-Zirconium-Titanium.

At least part of the support member may also comprise any one of the following combination of metals: Ag-Cd 44/49 at. % Cd; Au-Cd 46.5/50 at. % Cd; Cu-Al-Ni 14/14.5 wt. % Al and 3/4.5 wt. % Ni, Cu-Sn approx. 15 at. % Sn, Cu-Zn 38.5/41.5 wt. % Zn, Cu-Zn-X (X=Si, Al, Sn), Fe-Pt approx. 25 at. % Pt, Mn-Cu 5/35 at. % Cu, Pt alloys, Co-Ni-Al, Co-Ni-Ga, Ni-Fe-Ga, Ti-Pd in various concentrations, Ni-Ti (˜55% Ni). It will be appreciated that the foregoing list is provided merely as an example of suitable materials and is not an exhaustive list. The support member include alloys or other materials different from those provided above.

The shape memory material of the support member may act as a spine along the length of said support member.

At least part of the support member may be composed of a permeable material. Alternatively, at least part of the support member may be semi-permeable. In a further embodiment, at least part of the support member may be composed of an impermeable material.

The support member may be composed of polyether or polyester, polyurethanes or polyvinyl alcohol. The material may further comprise cellulose ranging from low to high density, having small, large, or twin pore sizes, and having the following features: closed or open cell, flexible or semi-rigid, plain, melamine, or post-treated impregnated foams. Additional materials for the support member can include polyvinyl acetal sponge, silicone sponge rubber, closed cell silicone sponges, silicone foam, fluorosilicone sponge. Specially designed structures using vascular graft materials such as PTFE, PET and woven yarns of nylon, may also be used.

The support member may further include semi-permeable membranes made from a number of materials. Example include polyimide, phospholipid bilayer, thin film composite membranes (TFC or TFM), cellulose ester membrane (CEM), charge mosaic membrane (CMM), bipolar membrane (BPM) or anion exchange membrane (AEM).

The support member may include at least a porous region to provide a matrix for tissue in-growth. Said region may further be impregnated with an agent to promote tissue in-growth.

The agent(s) released from the support member may comprise one or more of a large number of compounds and materials. Examples include but are not limited to any one or a combination of the following: adhesive materials, tissue growth promoting materials, sealing materials, drugs, biologic agents, gene-delivery agents, and/or gene-targeting molecules.

Adhesive agents include cyanoacrylates (including 2-octyl cyanoacrylate, n-butyl cyanoacrylate, iso-butyl-cyanoacrylate and methyl-2- and ethyl-2-cyanoacrylate), albumin based sealants, fibrin glues, resorcinol-formaldehyde glues (e.g., gelatin-resorcinol-formaldehyde), ultraviolet-(UV) light-curable glues (e.g., styrene-derivatized (styrenated) gelatin, poly(ethylene glycol) diacrylate (PEGDA), carboxylated camphorquinone in phosphate-buffered saline (PBS), hydrogel sealants, eosin based primer consisting of a copolymer of polyethylene glycol with acrylate end caps and a sealant consisting of polyethylene glycol and polylactic acid, collagen-based glues and polymethylmethacrylate, vascular endothelial growth factor, fibroblast growth factor, hepatocyte growth factor, connective tissue growth factor, placenta-derived growth factor, angiopoietin-1 or granulocyte-macrophage colony-stimulating factor.

Agents for modulating cellular behaviour include microfibrillar collagen, fibronectin, fibrin gels, synthetic Arg-Gly-Asp (RGD) adhesion peptides, tenascin-C, Del-1, CCN family (e.g., Cyr61) hypoxia-inducible factor-1, acetyl choline receptor agonists and monocyte chemoattractant proteins.

Gene delivery agents include viral vectors for gene delivery (e.g., adenoviruses, retroviruses, lentiviruses, adeno-associated viruses) and non-viral gene delivery agents/methods (e.g., polycation polyethylene imine, functional polycations, consisting of cationic polymers with cyclodextrin rings or DNA within crosslinked hydrogel microparticles, etc.).

Agents modulating cell replication/proliferation include target of rapamycin (TOR) inhibitors (including sirolimus, everolimus and ABT-578), paclitaxel and antineoplastic agents, including alkylating agents (e.g., cyclophosphamide, mechlorethamine, chlorambucil, melphalan, carmustine, lomustine, ifosfamide, procarbazine, dacarbazine, temozolomide, altretamine, cisplatin, carboplatin and oxaliplatin), antitumor antibiotics (e.g., bleomycin, actinomycin D, mithramycin, mitomycin C, etoposide, teniposide, amsacrine, topotecan, irinotecan, doxorubicin, daunorubicin, idarubicin, epirubicin, mitoxantrone and mitoxantrone), antimetabolites (e.g., deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate and aspariginase), antimitotic agents (e.g., vincristine, vinblastine, vinorelbine, docetaxel, estramustine) and molecularly targeted agents (e.g., imatinib, tretinoin, bexarotene, bevacizumab, gemtuzumab ogomicin and denileukin diftitox).

In one embodiment the one or more agents may comprise monoclonal antibodies. The monoclonal antibody may comprise anti-tumour properties. For example the monoclonal antibody may be an angiogenesis inhibitor such as Bevacizumab. The monoclonal antibody may also comprise anti-inflammatory properties.

Further examples of specific monoclonal antibodies include but are not limited to the following: Adalimumab, Basiliximab, Certolizumab pegol, Cetuximab Daclizumab, Eculizumab, Efalizumab, Gemtuzumab, Ibritumomab tiuxetan, Infliximab Muromonab-CD3, Natalizumab, Omalizumab, Palivizumab, Panitumumab, Ranibizumab, Rituximab, Tositumomab or Trastuzumab.

The agent(s) may be steroids such as corticosteroids, estrogens, androgens, progestogens and adrenal androgens.

Still further, the agent(s) may include antiplatelet, antithrombotic and fibrinolytic agents such as glycoprotein IIb/IIIa inhibitors, direct thrombin inhibitors, heparins, low molecular weight heparins, platelet adenosine diphosphate (ADP) receptor inhibitors, fibrinolytic agents (e.g., streptokinase, urokinase, recombinant tissue plasminogen activator, reteplase and tenecteplase, etc). Additionally, gene targeting molecules such as small interference RNA, micro RNAs, DNAzymes and antisense oliogonucleotides, or cells such as progenitor cells (e.g., endothelial progenitor cells, CD34+ or CD133+monocytes, hemopoietic stem cells, mesenchymal stem cells, embryonic stem cells, multipotent adult progenitor cells and inducible pluripotent stem cells) and differentiated cells (e.g., endothelial cells, fibroblasts, monocytes and smooth muscle cells) may be agent(s) 108. Furthermore, drug delivery agents like mucoadhesive polymers (e.g., thiolated polymers), or pharmacologic agents of local treatment of atherosclerosis such as high density lipoprotein cholesterol (HDL), HDL mimetics, heme oxygenase-1 inducers (e.g. probucol and its analogues, resveratol and its analogues), hydroxymethylglutaryl CoA (HMG-CoA) reductase inhibitors and fibrates (including fenofibrate, gemfibrozil, clofibrate etc) may be included agents.

In a further aspect, there is provided an apparatus for delivering an agent between an endoluminal prosthesis and a wall of a body lumen, the apparatus comprising:

a support member configured for placement between the prosthesis and the wall of the body lumen, wherein the support member includes a shape memory material changeable from an undeployed state to a deployed state; a capsule carried by the support member; and

an agent in the capsule.

In another aspect, there is provided an apparatus for delivering an agent between an endoluminal prosthesis and the wall of a body lumen, the apparatus comprising:

a generally conformable base portion extending around a periphery of the endoluminal prosthesis;

a single capsule within the base portion, wherein the capsule has a predetermined range of agent delivery pressures; and

an agent disposed in the capsule.

In a further aspect, there is provided a sealing device configured to act as an interface between an endoluminal prosthesis and a wall of a body lumen, the apparatus comprising:

a flexible support member composed of a shape memory alloy material, wherein the support member is changeable from (a) a first reduced profile configuration in which the support member is positioned for placement at a desired location, and (b) a second deployed configuration in which the support member extends concentrically between the wall of the vessel and the endoluminal device, and wherein the support member is not fixedly attached to an exterior surface of the endoluminal prosthesis;

a capsule carried by the support member; and

an agent in the capsule, wherein the capsule is configured to rupture at a predetermined range of pressures and release the agent.

The apparatus and endoluminal device of all aspects and embodiments disclosed herein may be used to seal an endoluminal prosthesis within a lumen. Said lumen include but are not limited to one or more of the following: cardiac chambers, cardiac appendages, cardiac walls, cardiac valves, arteries, veins, nasal passages, sinuses, trachea, bronchi, oral cavity, esophagus, small intestine, large intestine, anus, ureters, bladder, urethra, vagina, uterus, fallopian tubes, biliary tract or auditory canals.

In specific embodiments, the device may be used to seal a graft or stent within an aorta of a patient. In a further embodiment, the device may be used to seal an atrial appendage. In this embodiment, the device may deliver an agent to effect the seal of a prosthetic component across the opening to said atrial appendage.

In a further embodiment, the device of the present invention may be used to seal a dissection in a vessel. In this embodiment, the support member is positioned adjacent the opening of the false lumen and an intraluminal stent subsequently delivered thereto. Upon radial expansion of the stent, the support member is caused to release adhesive therefrom to seal the tissue creating the false lumen against the true vessel wall.

In a further embodiment, the device of the present invention is used to seal one or more emphysematous vessels.

In a still further embodiment, the device may be used to seal an artificial valve within a vessel of a subject. An example includes the sealing of an artificial heart valve. It is envisaged that the seal provided by the present device will prevent paravalvular leaks.

The endoluminal device may be configured such that it moves independently of the endoluminal prosthesis. Alternatively, the endoluminal device may be connected to said prosthesis for delivery to a target site. The endoluminal device may be connected to said prosthesis by any number of means including suturing, crimping, adhesive connection.

In a further embodiment, the endoluminal device may further include one or more engagement members. The one or more engagement members may include staples, hooks or other means to engage with a vessel wall thus securing the device thereto.

### BRIEF DESCRIPTION OF THE DRAWINGS

FIGS. 1A and 1B are partially schematic illustrations of a device configured in accordance with an embodiment of this disclosure.

FIGS. 2A-2F illustrate a method of deploying a device for delivering an agent between an endoluminal prosthesis and a wall of a body lumen in accordance with an embodiment of the disclosure.

FIGS. 3A-3C are partially schematic illustrations of a sealing device for delivering an agent between an endoluminal prosthesis and a wall of a body lumen in accordance with an embodiment of the disclosure.

FIGS. 4A and 4B are partially schematic illustrations of a sealing device for delivering an agent between an endoluminal prosthesis and a wall of a body lumen in accordance with another embodiment of the disclosure.

FIGS. 5A-5D are partially schematic illustrations of a portion of a sealing device configured in accordance with still another embodiment of the disclosure.

FIG. 6 is a partially schematic, isometric illustration of a portion of a pressure activated capsule or compartment configured in accordance with several embodiments of the disclosure.

FIGS. 7A and 7B are illustrations of a portion of a flexible support member configured in accordance with another embodiment of the disclosure.

FIGS. 8A and 8B are illustrations of a portion of a flexible support member configured in accordance with still another embodiment of the disclosure.

FIG. 9 shows a further embodiment of a support member of the disclosure.

### DETAILED DESCRIPTION

### A. Introduction

Aspects of the present disclosure are directed to endoluminal devices and associated systems and methods. In general, many of the techniques and associated devices described below include advancing an endoluminal prosthesis and sealing device through a body lumen in a first undeployed and reduced profile configuration. When positioned in situ, the sealing device of the present invention is capable of moving from its reduced radial profile configuration to a second configuration with an increased radial profile. In situ, and in its second configuration, the sealing device is configured to be positioned between the prosthesis and the wall of the body lumen. In one embodiment, when the endoluminal prosthesis is at the desired location in the body lumen, it is typically deployed from an introducer catheter whereupon it may move to an expanded radial configuration by a number of mechanisms. In some embodiments, the prosthesis may be spring expandable. Alternatively, a balloon or expandable member can be inflated within the lumen of the prosthesis to cause it to move to an expanded radial configuration within the vessel. This radial expansion, in turn, presses the sealing device against a wall of the body lumen until the sealing device releases an agent contained therein or thereon. Particularly, the expansion of the prosthesis may cause the rupture of a capsule of the sealing device to release agent contained within the capsule to the desired region. In several embodiments, the sealing device is configured to fully seal a proximal and/or distal end of the endoluminal prosthesis for endovascular aneurysm repair (EVAR) to prevent endoleaks and prevent subsequent migration and/or dislodgement of the prosthesis.

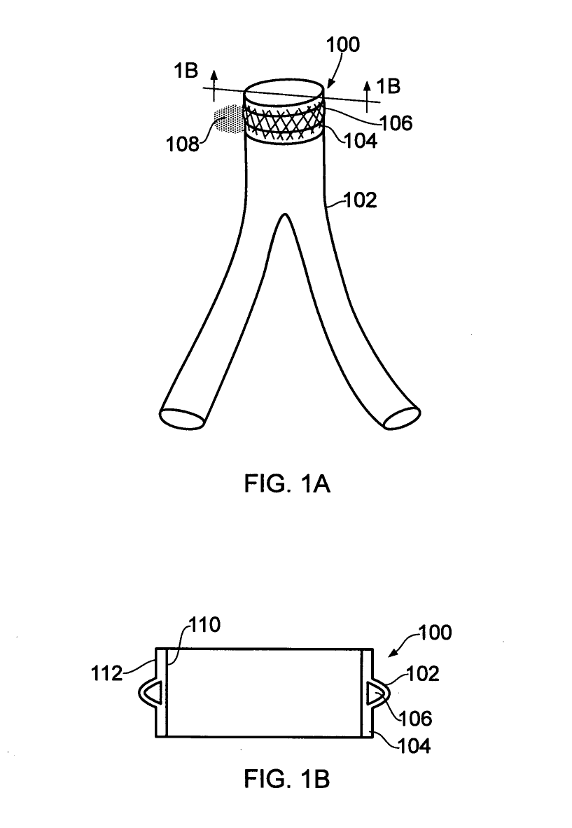

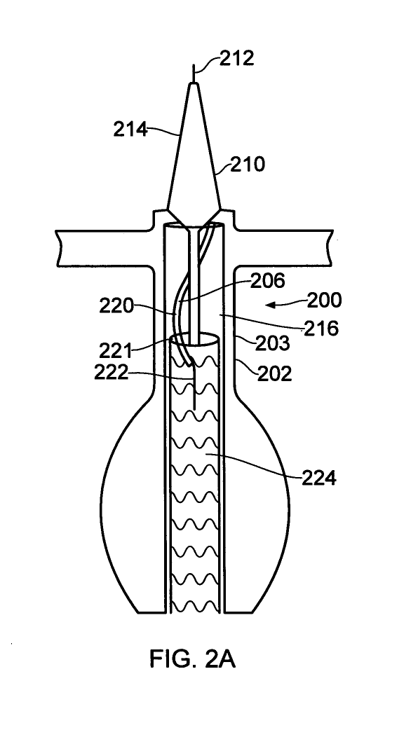

Many techniques and devices described in detail in one or more of the following sections may be combined with techniques and/or devices described in the same section and/or other sections. Several details describing devices or processes that are well-known to those of ordinary skill in the relevant art and often associated with such devices and processes are not set forth in the following description for purposes of brevity. Those of ordinary skill in the relevant art will understand that further embodiments may include features not disclosed in the following sections, and/or may eliminate some of the features described below with reference to FIGS. 1A-5D. Moreover, the particular features, structures, routines, steps, or characteristics described below may be combined in any suitable manner in one or more embodiments of this technology.

Where the context permits, singular or plural terms may also include plural or singular terms, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of features are not precluded.

### B. Embodiments of Endovascular Devices and Associated Systems and Methods

FIGS. 1A and 1B are partially schematic illustrations of an apparatus 100 for delivering an agent between an endoluminal prosthesis 102 and a wall of a body lumen (not shown) in accordance with an embodiment of this disclosure. More specifically, FIG. 1A is a partially schematic, isometric illustration of the apparatus 100 extending around a periphery of the endoluminal prosthesis 102, and FIG. 1B is a side, cross-sectional view taken substantially along lines 1B-1B of FIG. 1A. Referring to FIGS. 1A and 1B together, the apparatus 100 of this embodiment includes a generally conformable band or a containment band 104 extending around the periphery of the endoluminal prosthesis 102, a capsule or annular compartment 106 within the conformable band 104, and one or more agents or reactants 108 disposed in the capsule 106. The capsule 106 is configured to rupture at a predetermined range of pressures (e.g., 15-25 psi) and release the agent(s) 108.

In the illustrated embodiment, the apparatus 100 is proximate to an end of the endoluminal prosthesis 102. In other embodiments, however, the apparatus 100 may be positioned at a different location relative to the endoluminal prosthesis 102. Moreover, the apparatus 100 in the embodiment illustrated in FIGS. 1A and 1B is a separate, discrete component from the endoluminal prosthesis 102. In other embodiments, however, the apparatus 100 can be an integral component of the endoluminal prosthesis 102. It will be appreciated that the arrangement of the endoluminal prosthesis 102 of FIGS. 1A and 1B is merely shown as a representative arrangement of such a structure, and the endoluminal prosthesis 102 can have a variety of different lengths, diameters, and/or configurations.

The conformable band 104 can include a flexible component that is configured to conform to irregularities between the endoluminal prosthesis 102 and a vessel wall (not shown). As best seen in FIG. 1B, the conformable band 104 comprises a generally ring-like structure having a first or inner surface 110 and a second or outer surface 112. The conformable band 104 entirely surrounds the capsule 106 such that the capsule 106 is “suspended” within the conformable band 104. In other embodiments, however, the conformable band 104 can have a different shape and/or configuration.

The conformable band 104 can be composed of a permeable, semi-permeable, or impermeable material. It may be biostable or biodegradable. For example, the conformable band 104 may be composed of polyether or polyester polyurethanes, PVA, cellulose, ranging from low to high density, having small, large, or twin pore sizes, and having the following features: closed or open cell, flexible or semi-rigid, plain, melamine, or post-treated impregnated foams. Additional materials for the conformable band 104 can include polyvinyl acetal sponge, silicone sponge rubber, closed cell silicone sponges, silicone foam, fluorosilicone sponge. Specially designed structures using vascular graft materials including PTFE, PET, woven yarns of nylon, PP, collagen or protein based matrix may also be used.

The conformable band material may be used independently or in combination with a mesh made from shape memory alloys (as detailed below). Semi-permeable membranes may also be used, which can be made from the following materials: polyimide, phospholipid bilayer, thin film composite membranes (TFC or TFM), cellulose ester membrane (CEM), charge mosaic membrane (CMM), bipolar membrane (BPM), anion exchange membrane (AEM).

In one specific embodiment, for example, the conformable band 104 can comprise a porous material configured to prevent any embolization (distal or proximal) of released agent(s) 108 from the capsule 106. The conformable band may have a graded degree of relative porosity from relatively porous to relatively non-porous.

The conformable band 104 can further serve as a porous matrix for tissue in-growth and can aid in promoting tissue in-growth (e.g., by adding growth factors, etc.). This feature is expected to improve the long-term fixation of the endoluminal prosthesis 102. In another specific example, the conformable band 104 can be impregnated with activators (e.g., adhesive activator) that induce rapid activation of the agent (e.g., a tissue adhesive) after the agent 108 has been released from the capsule 106. In other embodiments, however, the conformable band 104 can be composed of different materials and/or include different features.

In the illustrated embodiment, the capsule 106 is a single annular compartment within the conformable band 104, and extends completely around the periphery of the endoluminal prosthesis 102. In other embodiments, however, the capsule 106 may include one or more additional compartments or sections, and may not extend completely around the endoluminal prosthesis 102. Moreover, the capsule 106 may or may not be contained within the conformable band 104, and can be positioned at a different location on the apparatus 100 relative to the conformable band 104. In addition, the capsule 106 can have a variety of different shapes and/or sizes depending upon the particular application, the agent(s) 108, the configuration of the endoluminal prosthesis 102, and a number of other factors.

The agent(s) 108 in the capsule 106 can include adhesive materials, tissue growth promoting materials, sealing materials, drugs, biologic agents, gene-delivery agents, and/or gene-targeting molecules. For example, the agent 108 may include one or more of the following: cyanoacrylates (including 2-octyl cyanoacrylate, n-butyl cyanoacrylate, iso-butyl-cyanoacrylate and methyl-2- and ethyl-2-cyanoacrylate), albumin based sealants, fibrin glues, resorcinol-formaldehyde glues (e.g., gelatin-resorcinol-formaldehyde), ultraviolet-(UV) light-curable glues (e.g., styrene-derivatized (styrenated) gelatin, poly(ethylene glycol) diacrylate (PEGDA), carboxylated camphorquinone in phosphate-buffered saline (PBS), hydrogel sealants-eosin based primer consisting of a copolymer of polyethylene glycol with acrylate end caps and a sealant consisting of polyethylene glycol and polylactic acid, collagen-based glues and polymethylmethacrylate, vascular endothelial growth factor, fibroblast growth factor, hepatocyte growth factor, connective tissue growth factor, placenta-derived growth factor, angiopoietin-1 or granulocyte-macrophage colony-stimulating factor.

The agent(s) 108 may also include agents for modulating cellular behavior in relation to bioprosthesis, such as microfibrillar collagen, fibronectin, fibrin gels, synthetic Arg-Gly-Asp (RGD) adhesion peptides, tenascin-C, Del-1, CCN family (e.g., Cyr61) hypoxia-inducible factor-1, acetyl choline receptor agonists and monocyte chemoattractant proteins. Additional agents 108 can include gene delivery agents, such as viral vectors for gene delivery (e.g., adenoviruses, retroviruses, lentiviruses, adeno-associated viruses) and non-viral gene delivery agents/methods (e.g., polycation polyethylene imine, functional polycations, consisting of cationic polymers with cyclodextrin rings or DNA within crosslinked hydrogel microparticles, etc.). Still further agents 108 could include agents modulating cell replication/proliferation, such as target of rapamycin (TOR) inhibitors (including sirolimus, everolimus and ABT-578), paclitaxel and antineoplastic agents, including alkylating agents (e.g., cyclophosphamide, mechlorethamine, chlorambucil, melphalan, carmustine, lomustine, ifosfamide, procarbazine, dacarbazine, temozolomide, altretamine, cisplatin, carboplatin and oxaliplatin), antitumor antibiotics (e.g., bleomycin, actinomycin D, mithramycin, mitomycin C, etoposide, teniposide, amsacrine, topotecan, irinotecan, doxorubicin, daunorubicin, idarubicin, epirubicin, mitoxantrone and mitoxantrone), antimetabolites (e.g., deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate and aspariginase), antimitotic agents (e.g., vincristine, vinblastine, vinorelbine, docetaxel, estramustine) and molecularly targeted agents (e.g., imatinib, tretinoin, bexarotene, bevacizumab, gemtuzumab ogomicin and denileukin diftitox).

Additionally, the agent(s) 108 may be steroids such as corticosteroids, estrogens, androgens, progestogens and adrenal androgens. Still further agents 108 may include antiplatelet, antithrombotic and fibrinolytic agents such as glycoprotein IIb/IIIa inhibitors, direct thrombin inhibitors, heparins, low molecular weight heparins, platelet adenosine diphosphate (ADP) receptor inhibitors, fibrinolytic agents (e.g., streptokinase, urokinase, recombinant tissue plasminogen activator, reteplase and tenecteplase, etc). Additionally, gene targeting molecules such as small interference RNA, mico RNAs, DNAzymes and antisense oliogonucleotides, or cells such as progenitor cells (e.g., endothelial progenitor cells, CD34+ or CD133+monocytes, hemopoietic stem cells, mesenchymal stem cells, embryonic stern cells) and differentiated cells (e.g., endothelial cells, fibroblasts and smooth muscle cells) may be agent(s) 108. Furthermore, drug delivery agents like mucoadhesive polymers (e.g., thiolated polymers), or pharmacologic agents of local treatment of atherosclerosis such as high density lipoprotein cholesterol (HDL), HDL mimetics and hydroxymethylglutaryl CoA (HMG-CoA) reductase inhibitors, may be included agents 108. In still further embodiments, the agent(s) 108 can include one or more different materials.

Referring back to FIGS. 1A and 1B together, in operation, the endoluminal prosthesis 102 and apparatus 100 are positioned intravascularly within a patient (not shown) so that the apparatus 100 is at a desired location along a vessel wall. A balloon or other expandable member (not shown) is then expanded radially from within the endoluminal prosthesis 102 to press or force the apparatus 100 against the vessel wall. As the balloon expands, the capsule 106 ruptures and the agent(s) 108 are released. In one specific embodiment, for example, the agent 108 comprises an adhesive material and when the capsule 106 ruptures, the adhesive material flows through the pores of the conformable band 104. As mentioned above, the conformable band 104 can control the flow of the adhesive to prevent embolization of the adhesive material.

The apparatus 100 is expected to provide several advantages over conventional endovascular graft assemblies. For example, in an embodiment wherein the apparatus 100 includes a singular capsule or annular compartment 106 of the apparatus 100, the shelf-life of the agent(s) 108 within the capsule 106 may be prolonged. One technical problem associated with storing many types of agents 108 (e.g., cyanoacrylate adhesives) in very small packets or compartments is that such materials are highly reactive with the encapsulation material. The single capsule or annular compartment 106 around the perimeter of the apparatus 100 has a lower ratio of surface area to volume than a plurality of individual compartments. The single annular compartment 106 of the apparatus 100 accordingly is expected to reduce the potential for reaction between the agent 108 and the encapsulation material to prolong the shelf-life of the agent 108.

Another feature of the abovementioned embodiment of apparatus 100 is that the single capsule or annular compartment 106 provides the ability to control the uniform circumferential rupturing of the capsule 106 upon radial expansion and reduces the profile of the endoluminal prosthesis 102 and apparatus 100 for delivery to the desired location within a delivery catheter. Still another feature of the apparatus 100 is that the conformable band 104 is well suited to conform to the contour of an aneurysm neck. This enables the agent 108 (e.g., an adhesive material) to conform to irregularities between the endoluminal prosthesis 102 and the aneurysm neck to obtain an effective, fluid-tight seal.

The conformable band may be made from a hydrogel material which expands in situ to provide a moldable band around the prosthesis.

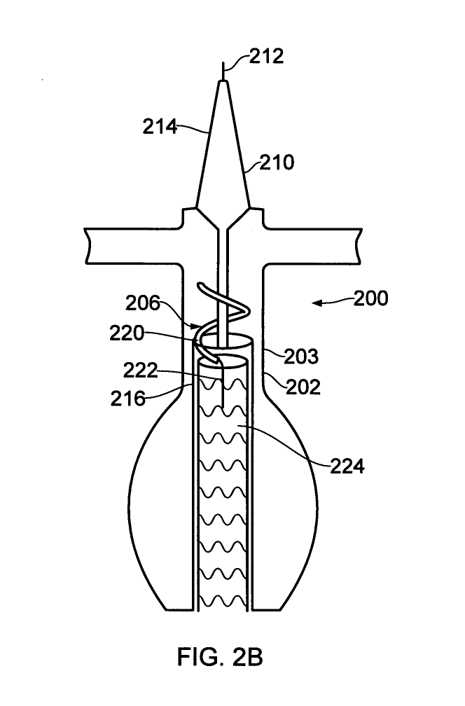

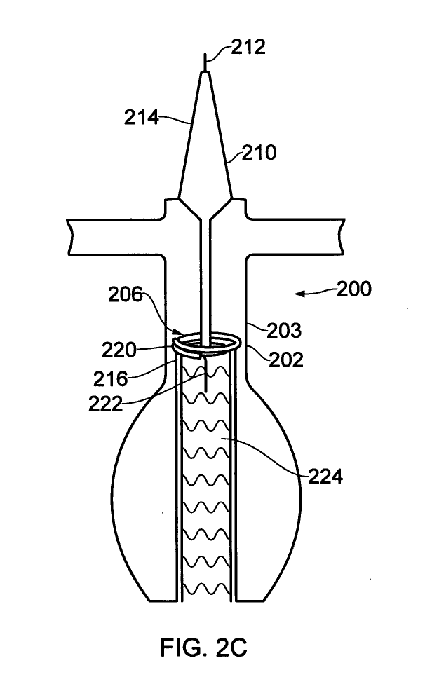

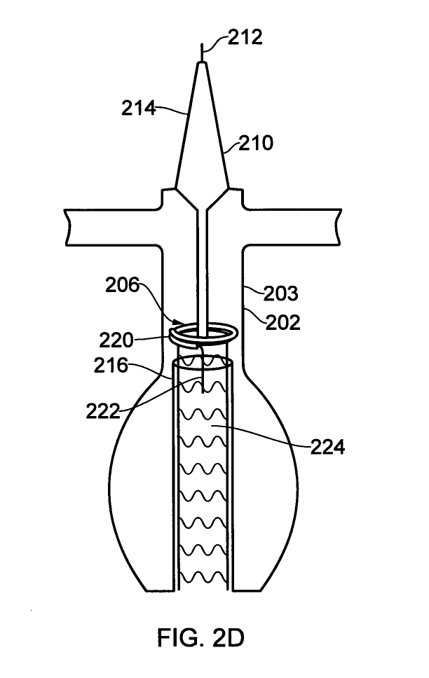

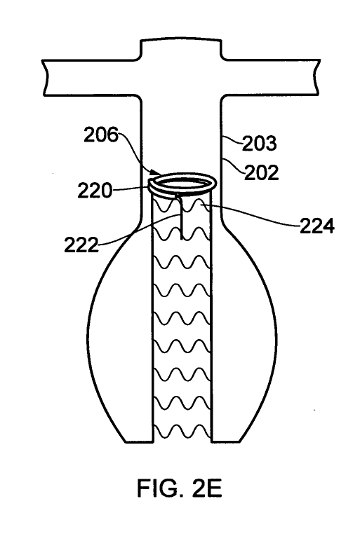

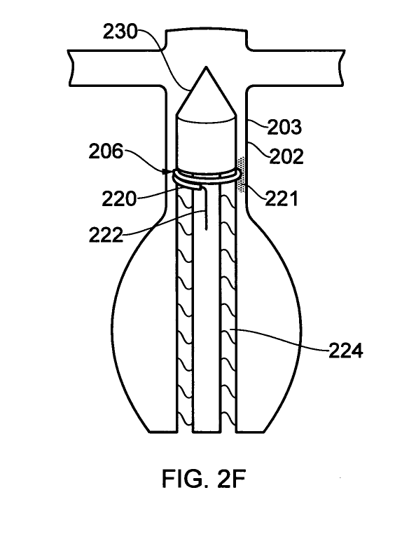

FIGS. 2A-2F are enlarged cross-sectional views illustrating a method of deploying an apparatus 200 for delivering an agent between an endoluminal prosthesis and a wall of a body lumen in accordance with an embodiment of the disclosure. More specifically, FIGS. 2A-2F illustrate a method of advancing the apparatus 200 into a desired location within the patient's vessel in a generally undeployed first configuration, and subsequently deploying the apparatus 200 to a second configuration to attach and seal the endoluminal prosthesis to the wall of the body lumen. Suitable body lumens can include one or more of the following: cardiac chambers, cardiac appendages including the atrial appendage, cardiac walls, cardiac valves, arteries, veins, nasal passages, sinuses, trachea, bronchi, oral cavity, esophagus, small intestine, large intestine, anus, ureters, bladder, urethra, vagina, uterus, fallopian tubes, biliary tract or auditory canals.

Beginning with FIG. 2A, a practitioner advances a delivery catheter 210 along a guide wire 212 to the desired location within the patient's vessel 202. The delivery catheter 210 can include, for example, a nose portion 214 and an introducer sheath 216. The configuration as shown in FIG. 2A enables the various components of the apparatus 200 (described in greater detail below) to be positioned independently of the endoluminal prosthesis, thus enabling a distribution of mass along the length of the delivery catheter 210. This in turn reduces the “packing density” or the volume of apparatus per unit length of the catheter 210. The reduction in the packing density is expected to significantly reduce the profile (or French size) of the delivery system and can also help reduce the deployment forces (e.g., due to reduction in the friction of the internal components), thereby increasing the ease-of-use for the physician. The reduction in profile can also enable the treatment of a large percentage of patients currently left untreated due to limitations in the size of the access vessels.

The apparatus 200 can include a sealing device 206 proximate to an end (proximal or distal) of the endoluminal prosthesis 224 (e.g., a stent graft) during deployment within the vessel 202 of the patient. The sealing device 206 can include, for example, a cylindrical capsule 220 integrated with a flexible support member 222. An agent 221 (e.g., adhesive material, etc.) is disposed within the capsule 220. The stent graft 224 is in a “crimped” or compressed state within the introducer sheath 216 at this stage of the process. The support member 222 is configured to act as the “spine” of the assembly. The support member 222 can include a shape memory material changeable from an undeployed or initial state (as shown in FIG. 2A) to a deployed or final state (as shown in FIG. 2E) in which the sealing device 206 is outside of the stent graft 224 and between the graft and a wall 203 of the vessel 202.

The support member 222 can be composed of a shape memory material such as Nickel-Titanium (nitinol wire), or shape memory alloys of the following combinations of metals: Copper-Zinc-Aluminium, Copper-Aluminium-Nickel, Copper-Aluminium-Nickel, Iron-Manganese-Silicon-Chromium-Manganese and Copper-Zirconium. Additionally, Titanium-Palladium-Nickel, Nickel-Titanium-Copper, Gold-Cadmium, Iron-Zinc-Copper-Aluminium, Titanium-Niobium-Aluminium, Uranium-Niobium, Hafnium-Titanium-Nickel, Iron-Manganese-Silicon, Nickel-Iron-Zinc-Aluminium, Copper-Aluminium-Iron, Titanium-Niobium, Zirconium-Copper-Zinc, Nickel-Zirconium-Titanium. The support member 222 may also be composed of the following combination of metals: Ag-Cd 44/49 at. % Cd; Au-Cd 46.5/50 at. % Cd; Cu-Al-Ni 14/14.5 wt. % Al and 3/4.5 wt. % Ni, Cu-Sn approx. 15 at. % Sn, Cu-Zn 38.5/41.5 wt. % Zn, Cu-Zn-X (X=Si, Al, Sn), Fe-Pt approx. 25 at. % Pt, Mn-Cu 5/35 at. % Cu, Pt alloys, Co-Ni-Al, Co-Ni-Ga, Ni-Fe-Ga, Ti-Pd in various concentrations, Ni-Ti (˜55% Ni). It will be appreciated that the foregoing list is provided merely as an example of suitable materials and is not an exhaustive list. The support member 222 may be composed of alloys or other materials different from those provided above.

The capsule 220 may be composed of polymeric and non-polymeric materials. Polymeric material may include LDPE, HDPE, PP, PTFE, silicone, or fluorosilicone. Other fluoropolymers that may be used for the construction of the capsule 220 include: PTFE (polytetrafluoroethylene), sold by DuPont under the trade name Teflon; sold by Solvay Solexis under the trade names Algoflon and Polymist, PFA (perfluoroalkoxy polymer resin), sold by DuPont under the trade name Teflon Hyflon, FEP (fluorinated ethylene-propylene), sold by DuPont under the trade name Teflon, ETFE polyethylenetetrafluoroethylene(Tefzel), (Fluon), PVF polyvinylfluoride (Tedlar), ECTFE polyethylenechlorotrifluoroethylene (Halar), PVDF polyvinylidene fluoride (Kynar, Solef, Hylar), PCTFE (Kel-F, CTFE) polychlorotrifluoroethylene, FFKM (Kalrez, Tecnoflon), FPM/FKM (Viton, Tecnoflon FKM), PFPE Perfluoropolyether (Fomblin, Galden), Nafion (Organofluorine, Organohalogen), EP (Fluorinated ethylene propylene), THV (terpolymer of tetrafluoroethylene, hexafluoropropylene and vinylidene fluoride), and PEEK. It may also comprise non-polymeric materials such as glass, bioglass, ceramic, platinum and titanium. It may further comprise biologically based materials such as crosslinked collagen or alginates. It will be appreciated that the foregoing list is provided merely as an example of suitable materials and is not an exhaustive list. The capsule 220 may be composed of a material or combination of materials different from those provided above.

Referring next to FIG. 2B, the practitioner begins retracting the introducer sheath 216, thereby exposing at least a portion of the apparatus 200. More specifically, as the introducer sheath 216 is retracted, the sealing device 206 is no longer radially confined and can begin to transition from the undeployed configuration in which the capsule 220 and support member 222 are generally straight to the deployed configuration in which the capsule 220 and support member 222 have a generally spiral or circular configuration. At this stage, the stent graft 224 is still in the compressed or crimped state within the introducer sheath 216.

In FIG. 2C, the introducer sheath 216 has completely released the sealing device 206, and the capsule 220 and corresponding portion of the support member 222 have moved into the deployed configuration in which the components have a generally circular or concentric arrangement. The stent graft 224 is still within the introducer sheath 216. Referring next to FIG. 2D, the stent graft 224 is pushed proximally from within the introducer sheath 216 such that a “seal zone” of the stent graft 224 is aligned with at least a portion of the sealing device 206.

Referring next to FIG. 2E, the stent graft 224 is expanded completely by fully retracting the delivery catheter 210 from the vessel 202. At this stage of the method, the capsule 220 is positioned between the stent graft 224 and the vessel wall 203. In FIG. 2F, the practitioner advances an inflatable member 230 (e.g., a balloon, etc.) through the vessel 202 until the inflatable member 230 is aligned with at least a portion of the capsule 220 and the “seal zone” of the stent graft 224. The capsule 220 is between the inflatable member 230 and the wall 203 of the vessel 202.

When the inflatable member 230 is inflated to a specified range of delivery pressures (e.g., with saline or another suitable inflation medium), the inflatable member 230 radially expands and presses the capsule 220 against the wall 203 until the capsule 220 ruptures and releases the agent 221. The capsule 220 is configured to release the agent 221 uniformly or at least approximately uniformly about the entire periphery of the stent graft 224. In a particular embodiment, the agent 221 includes an adhesive material, thereby sealing and securing the stent graft 224 to the vessel wall 203. In other embodiments, other types of agents or reactants can be delivered to the region.

One advantage of having the support member 222 composed of a shape memory material is that the sealing device 206 can be resheathed into the delivery catheter 210 and put back into an undeployed configuration if the deployment process is unsuitable, at an undesirable location, or otherwise needs to be repeated. Moreover, in certain embodiments, release of the agent 221 from the capsule 220 only occurs after the inflatable member 230 is inflated over a specified range of pressures. Accordingly, the apparatus 200 can be completely recoverable (for redeployment) if the inflatable member 230 is not so inflated.

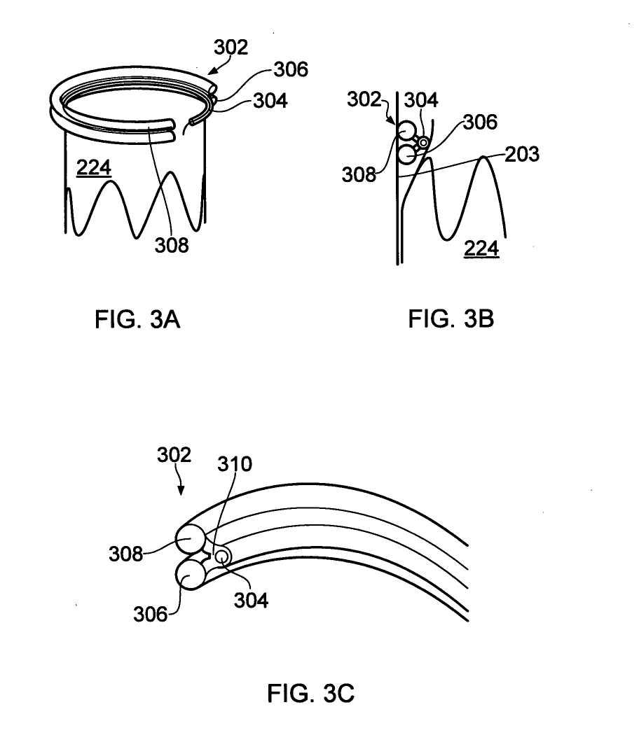

FIGS. 3A-5D are partially schematic illustrations of sealing devices for delivering an agent between an endoluminal prosthesis and a wall of a body lumen in accordance with another embodiment of the disclosure. The sealing devices described below with respect to FIGS. 3A-5D, for example, can be used with the apparatus 200 described above with reference to FIGS. 2A-2F, and can have many of the same features and advantages as the sealing device 206 described above. In other embodiments, however, the sealing devices described below can be used with other suitable assemblies and/or in other applications.

Referring to FIGS. 3A-3C, for example, a sealing device 302 can include a support member 304, a cylindrical capsule 306 carried by the support member 304, and a containment band 308 carried by the support member 304. An agent (not shown) is disposed within the capsule 306. As best seen in FIG. 3C, the support member 304, capsule 306, and containment band 308 are attached together via an attachment member 310. As with the sealing device 206 described above, the support member 304 is configured to act as the “spine” of the assembly and can include a shape memory material (e.g., nitinol wire) changeable from an undeployed or initial state to a deployed or final state (as shown in FIG. 3A) in which the sealing device 302 is outside of the stent graft 224 and between the graft and the vessel wall 203.

One feature of the containment band 308 in the sealing device 302 is that the containment band 308 can allow for removal of the endothelium layer during deployment of the device. In particular, the containment band 308 can remove all or at least a portion of the endothelium layer during deployment by performing a “scraping” action as the support member 304 goes from the undeployed state to the deployed state.

The containment band 308 is also expected to prevent or inhibit any agent (e.g., adhesive) particles from embolizing into the blood stream during the deployment or ballooning process or post-deployment. For example, with the release of an adhesive material from the capsule 306, part of the adhesive will polymerize along the containment band 308 and thereby form a reinforcing sealing layer. This is expected to enhance the sealing of the aneurysm acutely and help maintain the device long-term. Moreover, given the porous nature of the containment band 308, it is expected that tissue will grow over the band and form a reinforcing layer for the enhancement of both seal and fixation. This is expected to result in significant improvement of the long-term performance of the endoluminal prosthesis as compared with conventional arrangements.

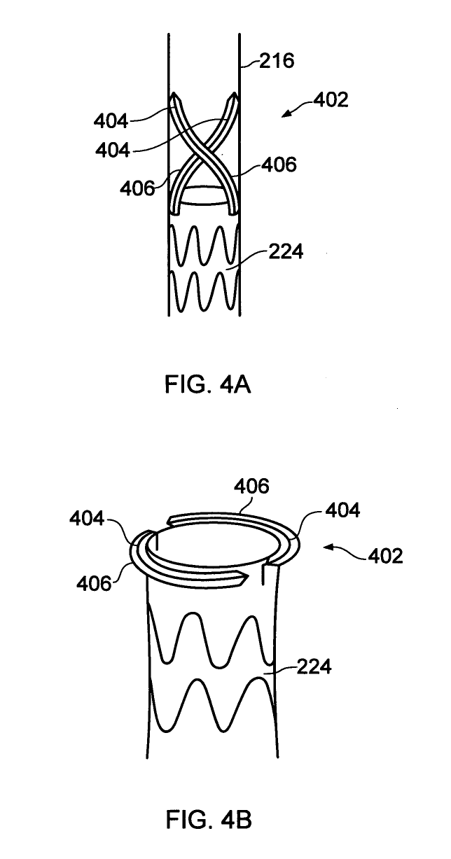

FIGS. 4A and 4B illustrate a sealing device 402 configured in accordance with still another embodiment of the disclosure. More specifically, FIG. 4A illustrates the sealing device 402 in an initial or undeployed configuration, and FIG. 4B illustrates the sealing device 402 in a deployed configuration. The sealing device 402 differs from the sealing devices 206 and 302 described above in that the sealing device 402 includes multiple support members 404. Each support member 404 (e.g., nitinol wire) carries a capsule 406. Although the sealing device 402 shown in FIGS. 4A and 4B includes two support members 404, the sealing device 402 can include a different number of support members 404.

One advantage of using multiple support members 404 is that this arrangement can help reduce the strain on each individual support member and enhance the performance of the sealing device 402. For example, the sealing device 402 can have more components attached to the individual support members 404 and the support members 404 can carry more weight. This feature is particularly useful when delivery of multiple agents may be necessary or when it is desirable to have more than one function at the site of interest. Another advantage of the sealing device 402 is that the use of multiple support members 404 can allow a reduction in the effective length of the individual support members 404. This feature is expected to result in quicker and potentially more accurate deployment, thereby saving critical procedural time.

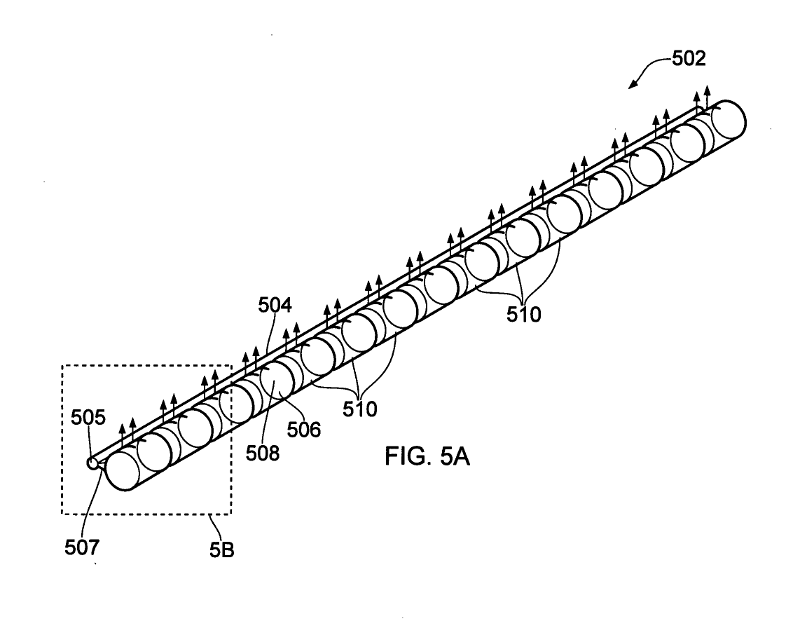

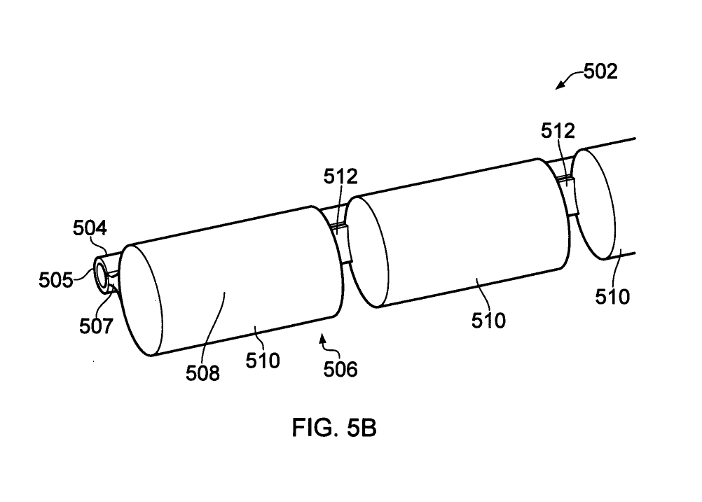

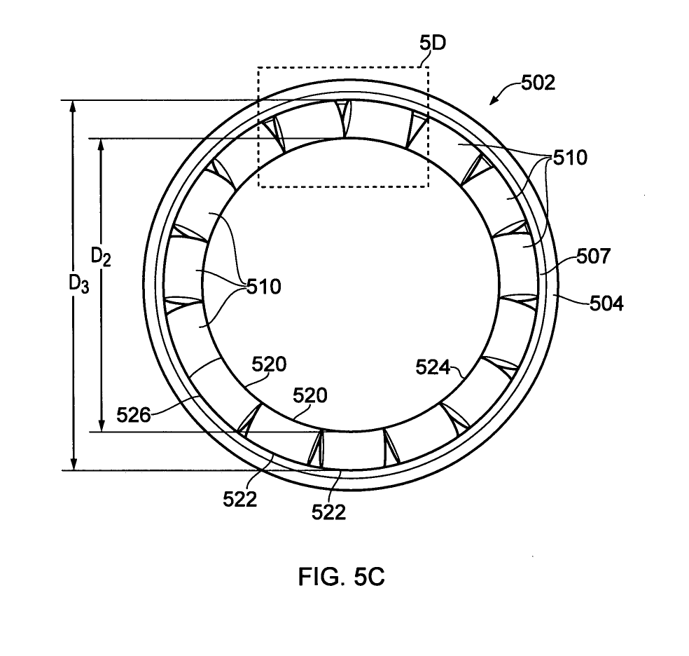

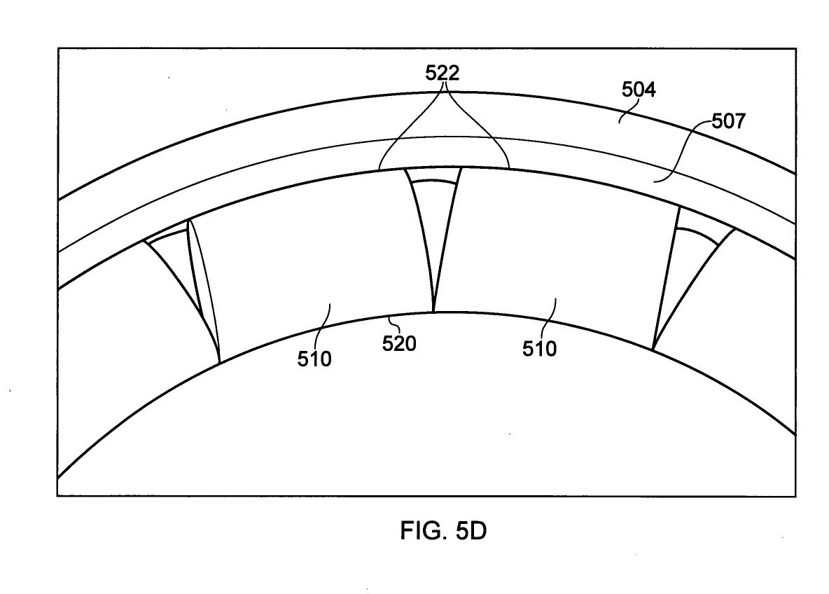

FIGS. 5A-5D are partially schematic illustrations of a portion of a sealing device 502 configured in accordance with still another embodiment of the disclosure. More specifically, FIGS. 5A is a partially schematic, isometric illustration of a portion of the sealing device 502, and FIG. 5B is an enlarged view of the area 5B of FIG. 5A. Referring to FIGS. 5A and 5B together, the sealing device 502 includes a flexible support member 504 and a capsule 506 carried by the support member 504. In the illustrated embodiment, the capsule 506 is attached to the support member 504 via a flexible attachment member 507. In other embodiments, however, the capsule 506 may be attached directly to the support member 504, or the capsule 506 may be attached to the support member 506 using an attachment member 507 having a different configuration. The support member 504 includes a lumen 505 that houses a shape memory material such as nitinol wire or the like (not shown). The lumen 505 can be sized based on the diameter of the shape memory material (e.g., the nitinol wire). An agent 508 is disposed within the capsule 506. The agent 508 can include one or more materials generally similar to the agent 108 described above with reference to FIG. 1. In other embodiments, the support member 504 and/or capsule 506 can have a different arrangement and/or include different features.

The sealing device 502 differs from the sealing devices described in that the capsule 506 includes a plurality of individual capsulets 510 carried by and extending lengthwise along the support member 504. The capsulets 510 are linked to each other with individual flex points or bend points 512. The flex points 512 are sections of reduced cross-sectional area that provide additional conformability and flexibility during the deployment process. The flex points 512 accordingly function as hinges and the individual capsulets 510 are configured to pivot relative to the respective flex points 512 and move close to each other when the support member 504 is driven from an undeployed configuration to a deployed configuration. In this way, the flex points 512 can help the sealing device 502 achieve a desired level of curvature in the deployed configuration, while minimizing the stress on the support member 504.

In the illustrated embodiment, the capsulets 510 are in fluid communication with each other. One feature of this arrangement is that during operation it can allow for a redistribution of pressure within the linked capsulets 510. This can help the capsule 506 release the agent 508 uniformly or at least approximately uniformly even in cases where pressure is applied to the sealing device 502 in a non-uniform fashion.

In other embodiments, the individual capsulets 510 are out of fluid communication with each other and each capsulet 510 contains a discrete volume of agent 508. In this case, the capsulets 510 are individually rupturable at a predetermined range of pressures (e.g., 15-25 psi). The capsulets 510 may each contain the same agent 508 or different agents or combinations of agents 508 may be disposed in the capsulets 510. Moreover, the capsulets 510 can be configured to rupture at the same ranges of pressures, or one set of capsulets 510 may be configure to rupture at a different range of pressures than a different set of capsulets 510.

As best seen in FIG. 5B, the individual capsulets 510 can have an outer dimension D of approximately 1 mm to 3 mm (e.g., about 2 mm). The outer dimension D can vary depending on a desired volume of agent 508 to be disposed in the capsulets 510, the particular application in which the sealing device 502 will be used, and a number of other factors. In one embodiment, the individual capsulets 510 and corresponding linkages 512 between the capsulets 510 comprise a single integrated unit formed. The single unit can be formed from a single piece of material or from two or more different material. In other embodiments, however, the capsulets 510 and the linkages 512 can be discrete, individual components that are attached together in the desired arrangement.

FIG. 5C is a partially schematic illustration of the sealing device 502 in a deployed configuration, and FIG. 5D is an enlarged view of the area 5D of FIG. 5C. Referring to FIGS. 5C and 5D together, the sealing device 502 can have a generally curved or concentric arrangement in the deployed configuration. Each capsulet 510 includes a first side 520 facing the support member 504 and a second side 522 facing away from the support member 504. The first sides 520 of the individual capsulets 510 define an inner circumference 524 having a first dimension D₂ and a generally continuous circular shape. The second sides 522 of the capsulets 510 define an outer circumference 526 having a second dimension D₃ larger than the first dimension D₂. One or more of the second sides 522 of the individual capsulets 510 are positioned to contact the wall of the vessel (not shown).

### C. Pressure Activated Capsules or Compartments and Methods for Forming Such Structures

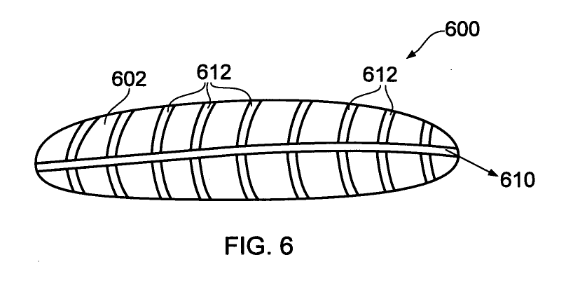

FIG. 6 is a partially schematic, isometric illustration of a portion of a pressure activated capsule 600 or compartment configured in accordance with several embodiments of the disclosure. The capsule 600 may be used with any devices described above with reference to FIGS. 1A-5D or with other suitable devices. The following discussion also outlines various techniques or processes for forming such the capsule 600 and other embodiments of pressure activated capsules or compartments.

The capsule 600 is configured to be carried by a support member (not shown) and an agent 602 can be disposed within the capsule 600. The capsule 600 also includes a stress concentration portion 610 extending lengthwise along an outer surface of the capsule 600. The stress concentration portion 610 can include, for example, a crack, stress point, or other type of failure point on the capsule 600 that will rupture when subjected to external pressure (e.g., from an inflatable member or balloon, such as the inflatable member 230 of FIG. 2F). This can enable rupturing of the capsule 600 within the limited exerted strain of 10 to 20% within the lumens of the body.

The capsule 600 can also include one or more strain restraining members or stiffening members 612 extending circumferentially about the capsule 600 and generally normal to the stress concentration portion 610. The stiffening members 612, for example, can include ribs or supports positioned to inhibit or minimize any extension of the capsule 600 in the circumferential direction when the capsule 600 is subjected to the external pressure (e.g., from the inflatable member). In this way, the stiffening members 612 serve as “strain constraints” and focus or direct the exerted strain on the stress concentration portion 610. The stiffening members 612 are an optional component that may not be included in some embodiments. In still other embodiments, the capsule 600 can have a different configuration and/or include different features.

A variety of different techniques or processes can be used to form pressure activated capsules or compartments (e.g., the capsule 600). The methods described below can be used to form pressure activated capsules or compartment suitable for use with any of the devices described above with reference to FIGS. 1A-5D, or with other suitable devices. In one particular embodiment, for example, a process for forming a pressure activated capsule can include pre-stressing the capsule during formation. The pre-stressed material will have a limited capacity to stretch when subjected to external pressure, and will fail when reaching critical stress on the stress-strain curve. The first stage of this method includes selecting a biocompatible capsule material that is also compatible with its contents (e.g., the agent 602 which can include adhesive material or a wide variety of other types of materials). The capsule material should also have a tensile strength suitable for the particular application in which the capsule will be used.

The next stage of this method includes forming an undersized capsule. The undersized capsule is essentially shaped as an extruded, elongated tube (e.g., a “sausage”) with one end of the tube sealed (e.g., by dipping, dip molding, vacuum forming blow molding, etc.). The process continues by expanding the capsule to its final shape. The capsule can be expanded, for example, by stretching (e.g., either hot or cold) using appropriate tooling so that the capsule material is pre-stressed to within a stress level, and whereby the clinical relevant balloon inflation pressure will exceed the failure stress of the capsule material. The method can further include filling the capsule with the desired contents while the capsule is under pressure so as to achieve pre-stressing in a single step. After filling the capsule, the capsule can be sealed (e.g., using a heat welding process, laser welding process, solvent welding process, etc.).

In another particular embodiment, a capsule can be formed by forming an air pillow or bubble wrap-type capsule assembly using a vacuum form process or other suitable technique. The next stage of this process includes perforating a film at the base of the capsule assembly and filling the individual capsules with the desired contents under an inert atmosphere. After filling the capsules, the puncture hole can be resealed by application of another film over the puncture hole and localized application of heat and/or solvent. In other embodiments, other methods can be used to seal the puncture hole. In several embodiments, the capsule can be configured such that the puncture hole re-ruptures at the same pressure as the capsule itself so that there is some agent (e.g., adhesive material within the capsule) flowing onto the corresponding portion of the endoluminal prosthesis.

In still another particular embodiment, one or more failure points can be created within a capsule. This process can include creating a capsule shaped as an extruded, elongated tube with one end of the tube sealed (e.g., by dipping, dip molding, vacuum forming blow molding, etc.). The capsule can be composed of a polymer material (e.g., polyethylene, polypropylene, polyolefin, polytetrafluoroethylene/Teflon families, and silicone rubber) or another suitable material. At one or more predetermined locations along the elongated tube, the process can include creating areas of substantially reduced thickness. These areas can be formed, for example, using a tool (e.g., a core pin with a razor blade finish along the length of the capsule), laser ablation, creating partially penetrating holes, creating an axial adhesive joint (e.g., tube from a sheet) that is weaker than the substrate, or other suitable techniques. The method next includes filing the capsule with the desired contents at a pressure below that required to rupture the thinned or weakened areas. After filling the capsule, the open end of the capsule can be sealed using one of the welding processes described above or other suitable processes.

In yet another particular embodiment, one or more stress points can be created within a capsule. This method can include forming a capsule and filling the capsule with the desired contents using any of the techniques described above. After forming the capsule and with the capsule in an undeployed configuration, the process can further include wrapping a suture (e.g., a nitinol wire) about the capsule at a predetermined pitch and tension. When the capsule is moved from the undeployed state to a deployed configuration and takes on a curved or circumferential shape, the suture compresses the capsule at the predetermined points. Stress points are created in the capsule walls at these points because of the increased pressure at such points.

In another embodiment the device may include one or more pressure points on the supporting member such as spikes or other raised areas which cause the penetration of the capsule once a predetermined pressure is applied thereto.

Still yet another particular embodiment for forming a pressure activated capsule or compartment includes creating a double walled capsule in which an inner compartment of the capsule is sealed and separated from an outer compartment of the capsule that contains the adhesive or other desired agent. The inner compartment can be composed of a compliant or flexible material, and the outer compartment can be composed of a substantially less compliant material. The outer compartment may or may not have failure points. The inner compartment is in fluid communication via a one way valve with a low compliance reservoir. The reservoir is configured to be pressurized by inflation of an expandable member or balloon to a high pressure, thereby allowing the valve to open and pressurize and expand the inner compartment. This process in turn pressurizes the outer compartment (that contains the adhesive) until the outer compartment ruptures. One advantage of this particular embodiment is that it can increase the pressure within the capsule to a value higher than otherwise possible with an external expandable member or balloon alone.

In a still further embodiment, the capsule has an inner compartment made from a relatively rigid material and an outer compartment made from a relatively flexible material. In this embodiment, the inner compartment acts as a reservoir, containing the agent and is designed to break or rupture at a predetermined pressure. The outer compartment may also have a failure pressure point to allow release of the agent. The rigidity of the inner compartment may provide a longer-term stability and shelf life of the encapsulated agent.

The application of rupture pressure may be carried out either locally or remotely, e.g. via a tube directly connected to the capsule that is connected to an external source at the delivery device entry site (e.g. femoral artery).

### D. Additional Embodiments of Flexible Support Members and Associated Systems and Methods

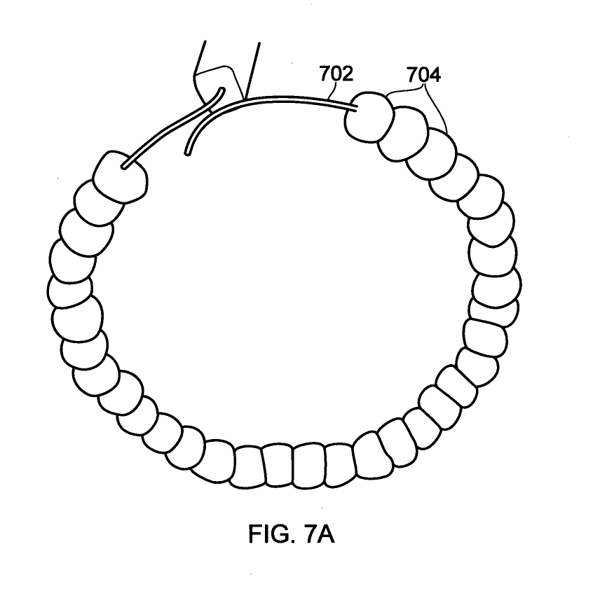

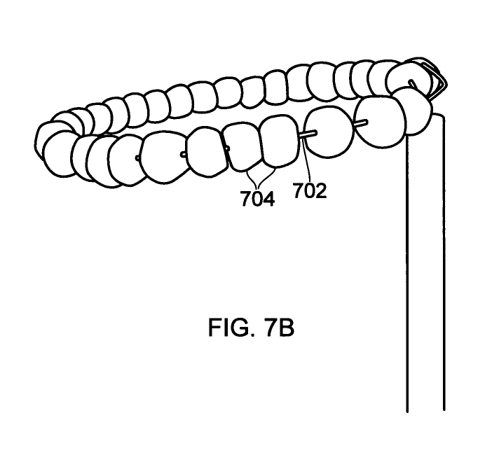

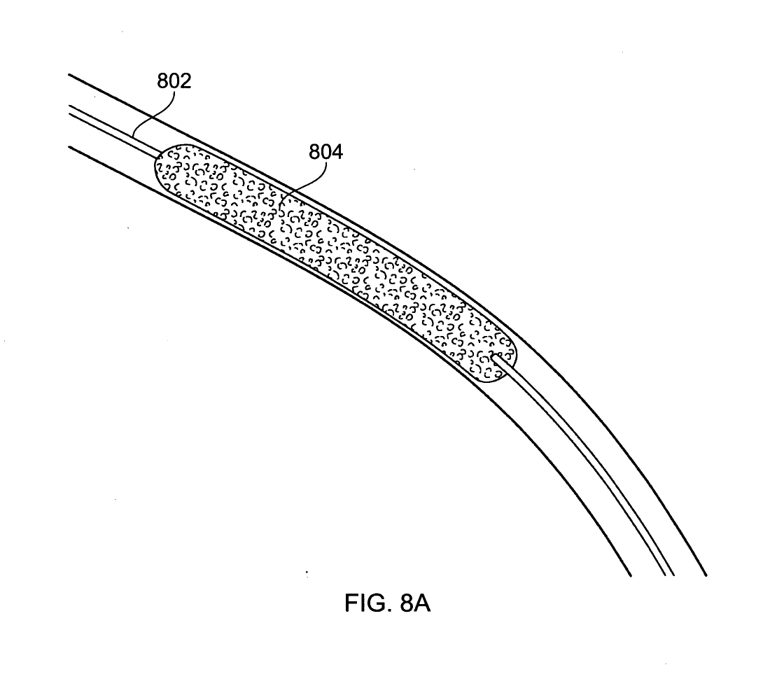

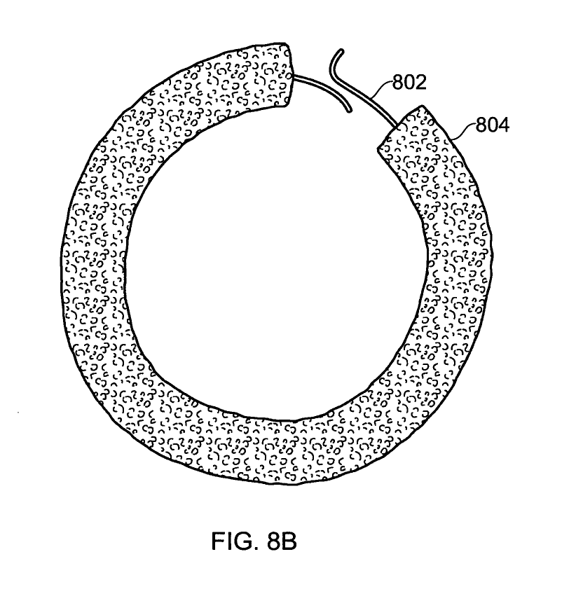

FIGS. 7A-8B are illustrations of flexible support members configured in accordance with additional embodiments of the disclosure. The flexible support members described below differ from those described above in that the support members of FIGS. 7A-8B are delivery systems configured to carry components or devices other than capsules containing agents. The flexible support members described below with respect to FIGS. 7A-8B can be used with any of the devices described above with reference to FIGS. 1A-6, and can have many of the same features and advantages as the flexible support members described above. In other embodiments, however, the flexible support member described below can be used with other suitable assemblies and/or in other applications. Examples of such applications are described in further detail below.

FIGS. 7A and 7B for example, illustrate a flexible support member 702 carrying a plurality of structural elements or features 704. The flexible support member 702 can be composed of shape memory materials generally similar to the shape memory materials described above (e.g., nitinol wire, etc.) and is configured to move from an undeployed or initial state to a deployed or final state (as shown in FIGS. 7A and 7B in which the support member 702 has a circumferential configuration. The structural elements 704 can include a wide variety of different suitable materials or elements (e.g., reinforcement elements to reinforce a location at which the device is deployed, elements to carry out a particular function at the deployment site, etc.). In other embodiments, the flexible support member 702 and/or the structural elements 704 can have a different arrangement or include different features.

FIGS. 8A and 8B illustrate a flexible support member 802 configured in accordance with still another embodiment of the disclosure. More specifically, FIG. 8A illustrates the flexible support member 802 in an initial or undeployed configuration, and FIG. 8B illustrates the support member 802 in a deployed configuration. In this embodiment, the support member 802 is carrying a scraper component 804 (e.g., a generally rough sandpaper-like component, a component having a straight “knife” edge, etc.). The flexible support member 802 can be composed of shape memory materials generally similar to the shape memory materials described previously (e.g., nitinol wire, etc.). In one particular example, as the flexible support member 802 moves from an undeployed state (FIG. 8A) to a deployed state in which the support member has a curved or circumferential configuration (FIG. 8B), the scraper component 804 can be used to perform a “vessel scrape,” an endothelium-denuding process, plaque removal, etc. The scraper component 804 can include a wide variety of different types of materials selected, at least in part, on the particular application for which the component will be used. In other embodiments, the flexible support member 802 and/or the scraper component 804 can have a different arrangement or include different features.

In other embodiments, the flexible support members 702 and 802 described above can be used to carry other types of devices or materials. For example, the support members can have active or passive coatings (e.g., drugs, growth factors, etc.) or other types of materials disposed along desired portions of the support member. In another specific example, the flexible support member 702 can be used to carry carbon nanotubes and deploy micro-nanomachines (e.g., microrobots) into the wall of the lumen. For example, the microrobots can deliver a poorly soluble or biological drug into deeper tissue layers or to a specific depth within the wall or through the wall. In still other embodiments, the flexible support members can be used to carry other types of materials and/or components.

One advantage of the flexible support members 702 and 802 described above is that by reducing the mass per unit volume inside the catheter, these devices are expected to significantly reduce the profile of a desired component or components for delivery to a desired location within a patient. This feature can be useful for in vivo assembly of devices in situations where the devices are composed of multiple components and it would be practically implausible to introduce them in the body percutaneously. This feature is also useful when it is desirable to have more than one function at the site of interest. This feature is further expected to result in quicker and potentially more accurate deployment of desired components or materials, thereby saving critical procedure time.

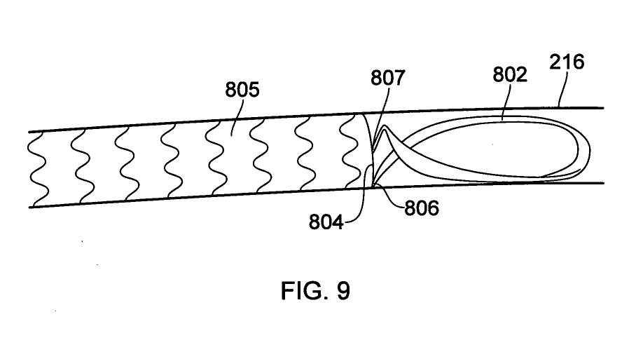

In the embodiment shown in FIG. 9, the device comprises a flexible support 802 which forms a looped configuration extending from a distal end 804 of graft 805. The support 802 is attached to the graft 805 at regions 806 and 807. The depiction in FIG. 9 shows the support member in its reduced radial profile configuration. Once in situ, the support member expands and substantially surrounds a region of the graft 805 at or adjacent distal end 804.

In all embodiments, the support member may be connected to a graft or stent by a tethering member. The tethering member may be made of an elastomeric material. Alternatively, the tethering member may be non-elastomeric and have a relatively fixed length.

### E. Conclusion

From the foregoing, it will be appreciated that specific embodiments of the disclosure have been described herein for purposes of illustration, but that various modifications may be made from these embodiments. Certain aspects of the disclosure described in the context of particular embodiments may be combined or eliminated in other embodiments. For example, a sealing device in accordance with particular embodiments may include only some of the foregoing components and features, and other devices may include other components and features in addition to those disclosed above. Further, while advantages associated with certain embodiments have been described in the context of those embodiments, other embodiments may also exhibit such advantages, and not all embodiments need necessarily exhibit such advantages. Accordingly, the disclosure can include other embodiments not shown or described above.

1. An endoluminal device for delivering an agent to a vessel of a subject, said endoluminal device comprising: at least one flexible support member configured for placement at least partially between an endoluminal prosthesis and a wall of a body lumen; at least one agent carried by the support member; said support member being changeable between a first relatively reduced radial configuration and a second relatively increased radial configuration; wherein in said first reduced radial configuration, the support member comprises an elongate member having a length which extends a distance from a first end to a second end; and wherein in said second increased radial configuration, said distance between said first end and said second end is relatively reduced.

2. The endoluminal device of claim 1 wherein the support member includes a shape memory material.

3. The endoluminal device of claim 1 wherein in said second increased radial configuration, the support member extends about a perimeter of the prosthesis.

4. The endoluminal device of claim 1 wherein said support member includes a capsule said capsule holding said at least one agent.

5. The endoluminal device of claim 1, wherein said support member further comprises a conformable band.

6. The endoluminal device of claim 5 wherein the conformable band comprises a generally porous material or a semi-porous material.

7. The endoluminal device of claim 5 wherein said conformable band comprises both relatively porous and relatively non-porous regions.

8. The endoluminal device of claim 1 wherein said at least one agent is releasable from said support member.

9. The endoluminal device of claim 1 comprising two or more differing agents.

10. The endoluminal device of claim 9 wherein the device includes a two part adhesive material wherein a first agent and a second agent are isolated from each other until release of one or both of the agents.

11. The endoluminal device of claim 1 wherein said at least one agent is released by the application of pressure to the support member.

12. The endoluminal device of claim 11 wherein the agent is released from the support member by the inflation of a balloon to cause the support member to compress against a wall of the vessel.

13. The endoluminal device of claim 1 being independently movable relative to said prosthesis.

14. The endoluminal device of claim 1 wherein the endoluminal prosthesis is for endovascular aneurysm repair.

15. The endoluminal device of claim 1 comprising a plurality of support members.

16. An endoluminal device including an apparatus for delivering an agent between an endoluminal prosthesis and a wall of a body lumen, the apparatus comprising: a support member configured for placement between the prosthesis and the wall of the body lumen, wherein the support member includes a shape memory material changeable from an undeployed state to a deployed state; a capsule carried by the support member; and an agent in the capsule.

17. The endoluminal device of claim 16 further including a capsule having a plurality of individual capsulets.

18. The endoluminal device of claim 17 wherein the individual capsulets are in fluid communication with each other.

19. The endoluminal device of claim 17 wherein the individual capsulets are not in fluid communication with each other and each capsulet contains a discrete volume of agent.

20. The sealing device of claim 17 wherein the capsulets are configured such that they each have individual predetermined ranges of pressures for release.

21. The sealing device of claim 17 wherein the individual capsulets and corresponding linkages between the capsulets comprise a single integrated unit.

22. An endoluminal assembly including: at least one support member; at least one agent carried by said support member, wherein said support member is changeable between a first relatively reduced radial configuration and a second relatively increased radial configuration; and wherein in said first reduced radial configuration, the support member comprises an elongate member having a length which extends a distance from a first end to a second end; and wherein in said second increased radial configuration, said distance between said first end and said second end is relatively reduced; said assembly further including a delivery means configured to hold said support member in said first reduced radial configuration, said delivery means also configured to deliver said endoluminal prosthesis to a target site in a vessel; wherein said at least one support member of the assembly is configured for placement at least partially between said endoluminal prosthesis and a wall of a body lumen.

23. A method for delivering at least one agent between an endoluminal prosthesis and a wall of a body lumen, the method comprising: advancing a sealing device to a desired location in the body lumen, said sealing device comprising a support member and at least one agent carried by the support member; causing or allowing said support member to change from a first relatively reduced radial configuration to a second relatively increased radial configuration,
wherein in said second increased radial configuration said support member defines a receiving region to receive at least a portion of the endoluminal prosthesis; advancing the endoluminal prosthesis to a desired location wherein at least part of the prosthesis is received in said receiving region of said support member; positioning an expandable member within a lumen of the endoluminal prosthesis and radially expanding the expandable member to exert a force on said support member; wherein said force causes the release of said agent from said support member.

24. A method for delivering an agent between an endoluminal prosthesis and a wall of a body lumen, the method comprising: advancing the endoluminal prosthesis to a desired location in the body lumen, wherein the endoluminal prosthesis includes a sealing device positioned between the prosthesis and the wall of the body lumen, and wherein the sealing device includes (a) a support member including a shape memory material, and (b) a capsule carried by the support member; positioning an expandable balloon in the body lumen with the sealing device between the balloon and the wall of the body lumen; and radially expanding the balloon to press the sealing device against the wall of the body lumen until the capsule releases an agent contained within the capsule.
